(19) United States
(12) Patent Application Publication
(10) Pub. No.: US 2020/0114036 A1
(43) Pub. Date: Apr. 16, 2020

(54) Liquid air freshener and delivery system

(71) Applicants: Sven Dobler, Huntington, NY (US); Per Dobler, Huntington, NY (US); Valeriano Marinelli, Port Washington, NY (US)

(72) Inventors: Sven Dobler, Huntington, NY (US); Per Dobler, Huntington, NY (US); Valeriano Marinelli, Port Washington, NY (US)

(21) Appl. No.: 16/350,227

(22) Filed: Oct. 16, 2018

(51) Int. Cl. A61L 9/12; B65D 75/52; B65D 75/22; A61L 9/01

(52) U.S. Cl. CPC A61L 9/127; A61L 9/01; B65D 75/225; B65D 75/52

(57) ABSTRACT

A liquid air freshener or delivery device to deliver ingredients from a liquid state to a vapor state, including a thermoformed container, having a flange, incorporates a window ply, through which fragrant material may be disseminated, having a paper or related ply thereunder, and which can absorb the liquid fragrance or ingredients when usage of the freshener or delivery is initiated. Below the paper ply is a seal layer, that seals the fragrance material or ingredients into the base of the container, until such time as the seal ply may be pierced, to allow the liquid fragrance or ingredients to migrate and saturate the paper ply, to initiate usage of the device. The base of the container, or the impregnatable paper ply, may have an integrally formed one or more piercing devices, that can pierce the seal layer, to initiate dissemination of the fragrance material or ingredients, and its scent or other properties, to the atmosphere.

### CROSS REFERENCE TO RELATED APPLICATION

This application is a non-provisional patent application to the provisional filed under Ser. No. 62/707,187, which was filed on Oct. 24, 2017.

### FIELD OF THE DISCLOSURE

This invention relates to a liquid air freshener, that integrates all of its various operative components into a single unit, that includes a fragrance material container itself, and a built in piercing system, in order to provide for a sustained release of the fragrance material, thereby eliminating the need for a separate injection molding holder, and discrete piercing apparatus, which substantially reduces the complexity of manufacture, usage, and cost.

This invention may also serve for any application other than air freshening such as for any diffusion of ingredients or chemicals into a vapor state, such as for example odor elimination, to attract or repel insects, animals, humans, or for therapeutic medical diagnostic applications.

### BACKGROUND

There are many air fresheners in the market. In their simplest form they are just scented paper (Car Freshener Corporation, “Little Tree”, Yankee Candle Jars “Car Jars”, AutoExpressions, etc). Other forms include scented gels (California Scents, Yankee Candle Car Gels, etc) and scented injection molded plastics.

The least expensive are typically the scented papers and the most expensive are the scented plastics.

Liquid membrane products can be more expensive also if they are often designed as cartridges to be applied to separately purchased injection-molded holders.

The disadvantage of scented paper is that it emits the fragrance more readily and lasts less as long as a packaged air freshener. This can be understood.

The advantage of a liquid membrane is that it includes more fragrance in a containment area that migrates through an active permeable membrane that allows a more prolonged time-release.

The problem with liquid active permeable membrane products is that the membrane selectively allows migration of some aroma chemicals more readily, and at a different rate than others. Some aroma chemicals are blocked from migrating altogether. Therefore the fragrances that work with liquid membranes are limited to prevent fragrance emission during shipment and storage. Furthermore, the membrane must be over-layered with a hermetic seal so that it does not start emitting fragrance through the permeable membrane prior to intended use. The user peels this membrane off, and then the membrane allows slow fragrance odor migration without any liquid materials passing through. The liquid membrane products are difficult to decorate or print, so often these devices are sold with optional holders or initiators which adds more costs.

A recent innovation in the market place such as the (P&G “Febreeze”) provides for an injection-molded holder assembly that includes a needle that upon flipping the attachment cam, pierces a seal layer on the integrated, pre-loaded thermoform cartridge and allows liquid fragrance material to pass through the perforation into a fragrance carrier material. This has allowed better performance and also a wider selection of aroma chemicals to be used. The great disadvantage is the need to manufacture thermoformed fragrance filled container with lidding, plus separately manufacture an injection-molded holder into which a mechanical piercing apparatus is then assembled and packaged, which add complexity and substantial cost in manufacturing. Also the fragrance carrier cannot be decorated or printed to help communicate branding information.

Published application No. US 2010/0314461, shows such a volatile composition dispenser, in the form of an apparatus, for use for incorporating a rupture element that incorporates a cam means for puncturing the volatile composition container for release of such a composition.

Published application No. US 2005/0127538, shows another apparatus type of device for providing for the evaporation of a volatile substance through a membrane.

Further United States patents show various apparatus for vaporizing aromas, such as perfumes, insecticides, or other volatile agents, as noted in U.S. Pat. No. 4,526,320. These are examples of related structures for disseminating the aromas of various materials.

All of the above air freshener devices, with the exception of the Febreeze product, and some scented gel that reduce in size over time, are also limited in that they do not provide an end of life use cue. A paper air freshener as well as a scented plastic looks the same when new and fresh as it does after it has given up its fragrance load and is no longer functional. The liquid permeable membrane air fresheners currently available in the marketplace do not provide an end of use cue as the fragrance container is designed as a cartridge and is either covered or hidden within the holder housing, or the container never empties fully, as the permeable membranes do not allow all solvents and aroma chemicals to pass.

### SUMMARY OF THE INVENTION

This invention contemplates the formation of a liquid air freshener or other device, that integrates all of its operative components together, incorporating a fragrance or liquid filled container, it may be a thermoform container made of heat formable material, typically a polyester. Alternatively, other plastic, injection molded, metal or other container, may also be used as long as the proposed design and adequate seal layer integrity can be provided and sustained until usage of the development.

The invention allows for an integrated, disposable, one part fragrance or ingredient delivery system. It provides a seal layer that contains the fragrance liquid odors or other absorbable ingredient intended to be dispersed into a vapor state (fragrance materials), without the need for a removable over-layer or holder. It also provides its own integral piercing system that is used to expose and migrate the fragrance material from its container, in preparation for usage, thus obviating the need for a separate injection molded holder and piercing apparatus, as normally required in the prior art. The invention allows multiple piercing and dosage steps that allows for a more prolonged timed release and user control of strength and longevity of application of the designed product. The currently available piercing membrane products upon the market allow for only a one time piercing at a preset location.

In order to allow for a much longer use of fragrance materials and/or aroma chemicals, and their dissemination, and therefore user choice and experience, this invention, as integrally formed, allows for selective usage, at timed intervals, at the selection of the user, during application. The invention also provides a much larger choice of use of ingredients and aroma chemicals, and therefore, adds to the user's choice and experience.

To allow for the carrier of this invention to be pre-scented, and act as a functional air freshener or delivery device prior to piercing of its seal layer, thus, this allows the user much greater control and longevity of application of the air freshener, than has previously been obtained. To allow for the carrier of this invention to be pre-scented or pre-dosed, and act as a functional air freshener prior to piercing its sealed layer, such provides for a much greater storage life, than heretofore obtained. Thus, to provide an end of life use cue, the fragrance material level drop will be visually apparent, through the thermoformed container of this invention, to let the user know when the fragrance material dissemination has been depleted. Thus, this invention provides an air freshener and delivery device, that improves the visual appeal, by including layers that may accept printed material, such as name, branding, instructions, and advertising, upon its fragrance carrier. Furthermore, the object of this invention is to provide a carrier that can be unscented at the time of manufacture and purchase, and the carrier can be printed, or unprinted, before its usage. The carrier can be predosed with fragrance material prior to its being inserted into the container during its assembly operations. The carrier can be dosed with fragrance material after insertion, but prior to the application of a window ply, and its attachment. And, the opening in the window ply can be sized to optimize the release performance of the freshener.

The invention further includes an integral sharp intrusion means, which after it has been activated, most of the fragrance material will be depleted, and at that time the user will know that the device has commenced to reach to the end of its life.

The container for the device or liquid air freshener can be made in other fashions, and can even be made of injection molded plastic, wood, metal or other materials. And, its various layers, in forming the container, can be heat sealed as the preferred means of attaching a seal layer and window ply to the container, although other means of attachment may be easily apparent to those skilled in the trade, for such application. For instance, adhesive sealing, sonic radio frequency sealing, and mechanical attachments, can be used.

Generally, the invention includes a thermoform container, that has a thermoformed flange applied to it, that has a window ply provided thereon, and various window openings which allows outflow of the scent of the fragrance material. It may then have a paper ply provided to its top, under the window ply, generally paper non-woven or other absorbent material, and it may have a polymer ply or other seal layer, to seal the top of the formed container.

Thus, the principal object of this invention is to provide a thermoformed container, or other container, for holding a liquid fragrance or viscous material, or even a flowable gel, that is manufactured to seal in the fragrance material initially, and since the container is formed or integrally attached as a thermoformed plastic container, it also has a sharp protrusion formed integrally or attached into its base, so that when it is forced upwardly, it ruptures the seal layer, and allows a saturation of the paper, non-woven layer or absorbent layer, to allow for dissemination of the fragrance material to the surrounding area, for scenting of the same, or for delivering ingredients into the vapor state during usage.

These and other objects may become more apparent to those skilled in the art upon review of the summary of the invention as provided herein, and upon undertaking a study of the description of its preferred embodiment.

### BRIEF DESCRIPTION OF THE DRAWINGS

FIG. 1 is a top view of the liquid air freshener of this invention;

FIG. 2 is a transverse sectional view taken through the embodiment of FIG. 1;

FIG. 2(a) provides an exploded view of the cross-section of the various components that make up the air freshener device of this invention;

FIG. 3 discloses schematically the means for activation of the air freshener, through the upward projection of an integral sharp protrusion, that breaks the seal layer, and applies the fragrance material to the paper layer for saturation and dissemination of fragrance material into the atmosphere vapor state;

FIG. 4 is an isometric view of the air freshener;

FIG. 5 is a bottom view, showing the location of the integrally formed sharp protrusion;

FIG. 6 shows modifications to the air freshener, and how it may include a string attachment, clip attachment, or have multiple sharp protrusions for sequential impaling the sealed layer;

FIG. 7 is a transverse view of the container shell of the invention, for holding the fragrance material, and disclosing the sharp protrusion integrally provided along its bottom wall;

FIG. 8 shows the process for making the liquid air freshener or device of this invention;

FIG. 9 shows the application of multiple protrusions for use for breaking the sealed ply, and including a supply of liquid integrally provided around the interior of its container shell;

FIG. 10 shows an alternative form of means for applying a perforation to the seal ply, where the piercing member is provided upon the paper, non-woven fragrance carrier;

FIG. 11 shows an alternative form of perforation member to facilitate the release of the fragrant material from its container shell and to the paper fragrance carrier; and

FIG. 12 A through C shows a wicking system to enhance the transfer of the fragrance or other material through the seal layer and into the absorbent layer.

### DESCRIPTION OF PREFERRED EMBODIMENTS

In referring to the drawings, and in particular FIG. 1, therein is shown the liquid air freshener 1 of this invention. It includes a thermoformed container, as noted, which includes an integral thermoform flange, it then has a window ply applied to the container, upon its flange, and the window ply has a series of openings, as noted. Then, a paper/non-woven or absorbent ply is applied to under the window ply, as can be noted. The paper ply is an absorbent form of paper or other absorbent material, so that when the fragrance material that is located within the container is allowed to migrate upwardly, to the paper ply, it saturates the same, to provide for the dissemination of its fragrance materials to the surrounding area/vapor state. Obviously, other materials may be used to form the various pieces, such as cardboard, paperboard, polymer piece thin wood, and related materials.

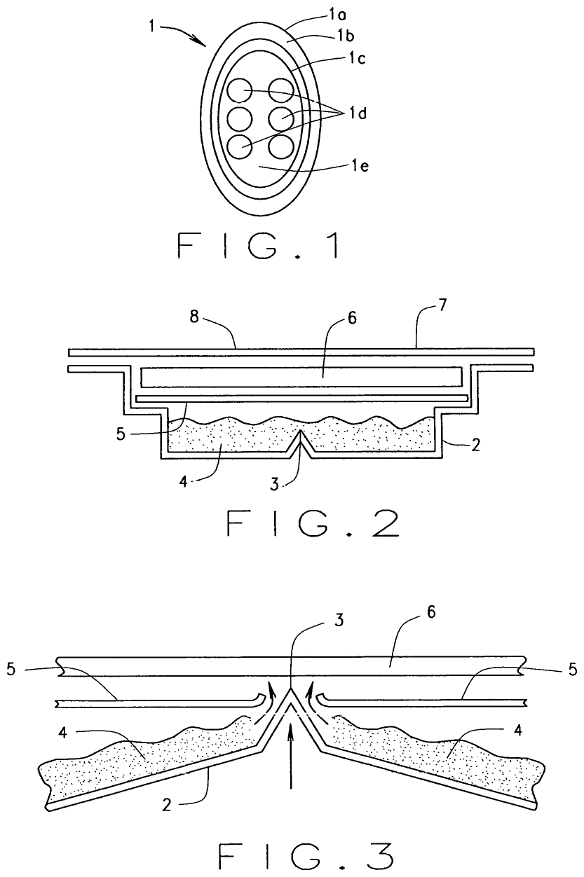

FIG. 2 provides a transverse sectional view through the FIG. 1 embodiment, and as can be seen, the thermoplastic container 2 has a raised sharp protrusion 3, integrally molded therein, in the base of the thermoformed container, and as can be seen, the container is disposed for holding the fragrance material 4 within its base. A seal layer 5 is provided, and seals the fragrance material within the container, until such time as the liquid air freshener is to be initiated, and used. Above the seal layer is the paper ply 6 and this paper ply is generally of a non-woven type, but this ply can be of any type of material that can absorb the fragrance material, when the seal layer has been pierced by the protrusion 3, to allow for the dissemination of the liquid of the fragrance material, that does infuse into the paper ply, and provides it with some degree of saturation, which then allows its aroma to escape into the ambient air, through the said window ply, during usage. A window ply 7 is provided on the top of the container, and it may have a series of window openings 8, as previously disclosed in FIG. 1, that allows for the scent of the fragrance to be released, but at the same time, prevents any contact with the fragrance saturated paper ply 6, as during usage of the device.

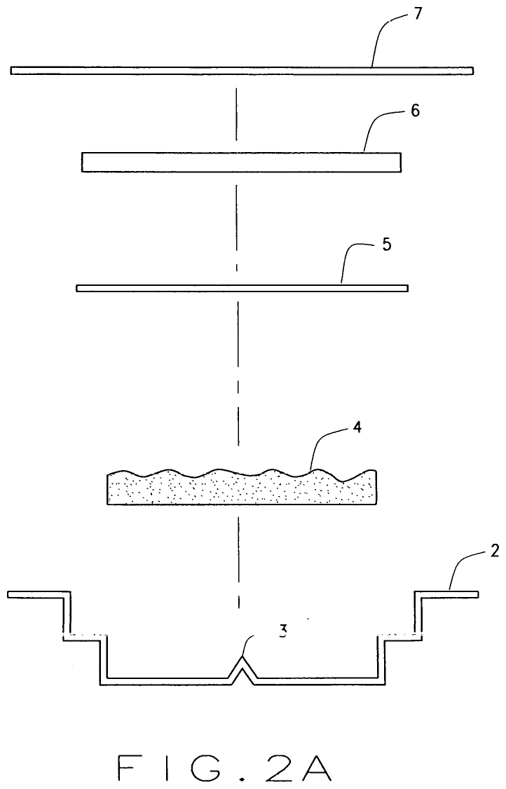

FIG. 3 provides a schematic view of the means for activation of the device/liquid air freshener of this invention. As noted, the paper/non-woven layer 6 is maintained above the bottom of the thermoformed container 2, and the bottom's integral sharp protrusion 3 will be pushed upwardly, from the bottom, through the stored fragrance material 4, and impale or pierce the seal layer 5, as can be noted. This allows for the sharp protrusion to project through the seal layer, and allows the fragrance material to migrate to be absorbed by the paper ply 6, in preparation for usage and application of the device/freshener. Thus, when the piercing of the seal layer has been done, that fragrance material will continue to wick into the paper/non-woven absorbent layer, to allow for continuance of release of the fragrance material to the surrounding area, during application and usage.

FIG. 4 provides a perspective view of the air freshener, and it can be seen that the upper window ply 7 does have a series of openings 8 provided therethrough, that furnishes a means for transfer of the fragrance material from the paper layer, out of the freshener, and into the ambient area.

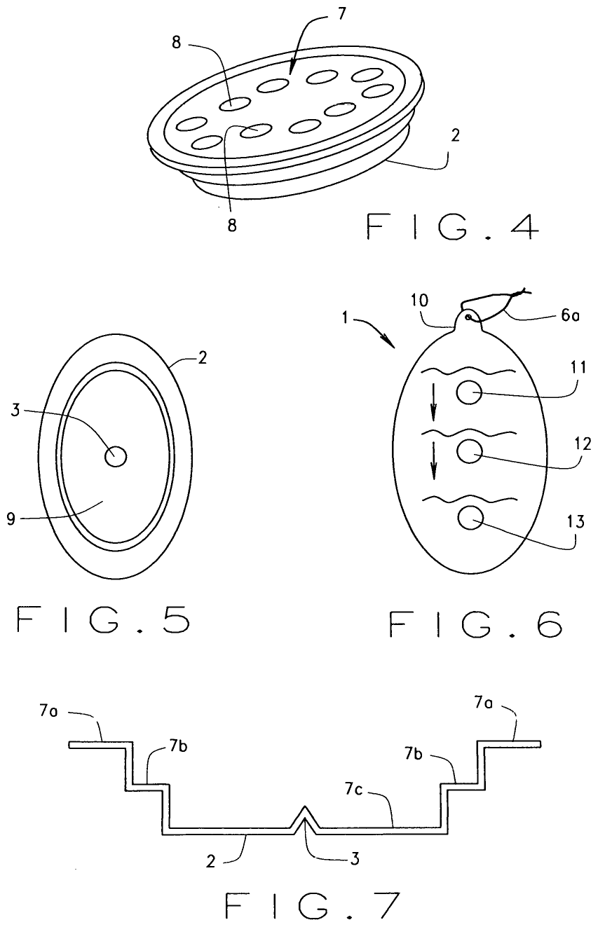

FIG. 5 provides a bottom view of the container, and as can be seen, the bottom of the container, as at 9, does have its integral sharp protrusion 3, with the container holding a supply of the liquid fragrance material as can be understood. Thus, when the user wishes to initiate the dissemination of the fragrance material, into the ambient atmosphere, one simply pushes the bottom of the container upwardly, until such time as its protrusion 3 pierces that seal layer, as previously explained. At that time, the fragrance material will be in contact with the bottom of the paper ply 6, and begin to infuse it with the fragrance material, for release to the atmosphere.

While it is not shown herein, there could also be a further seal ply applied to the top of the air freshener, to assure that no dissemination of its fragrance material will occur, until such time as it also may be peeled off, in preparation for application and usage of the freshener or device.

FIG. 6 shows some variations upon the air freshener 1 of this invention. In this instance, there may be other accessories attached to the container, such as providing an integral tab 10 to which a string attachment may be applied. Or, a form of clip attachment may be added, for the suspension of the freshener during its usage. Furthermore, as can be noted, there are three sharp protrusions 11 through 13, and these protrusions can be forced upwardly, separately, so that when the upper protrusion 11 pierces the seal ply or layer, it will allow the fragrance material at that level to flow to the underside of the paper ply 6, to initiate the release of a portion of the fragrance material. Then, when that is depleted, the second protrusion 12 can be used, to break the seal layer at that location, to allow some additional of the fragrance material to flow and contact the paper ply, for continued usage of the freshener. Then, the sharp protrusion 13 can be pierced, to emit its fragrance material. Thus, this will sustain the usage of the device, particularly when it is suspended upright, as noted in FIG. 6, through the use of its string or other attachments, as can be understood.

It might be commented at this time that various other carriers may be used, as scent carriers, and while the paper ply has been defined herein, it is just as likely that a sponge may form the layer 6, or natural fibers, synthetic fibers, cotton, wool, or cardboard, and any natural or man-made equivalent materials that can absorb and release the liquid fragrance material, during usage and application.

FIG. 7 shows a modification to the container shell 2 of this invention. It has various levels, as at 7a, 7b, and 7c, as noted. As can be seen, “a” provides the upper portion of the container 2, onto which the window ply is secured or sealed thereto. The b layer provides the base for attachment of the seal layer thereto. These layers are all provided for convenience of manufacture. And, the paper or absorbent layer 6 locates between the 7a layer and the 7b layer, as can be noted in FIG. 2. Finally, the 7c area provides the integrally formed piercing sharp protrusion 3, which when forced upwardly, breaks the seal layer for initiating the operations of the invention.

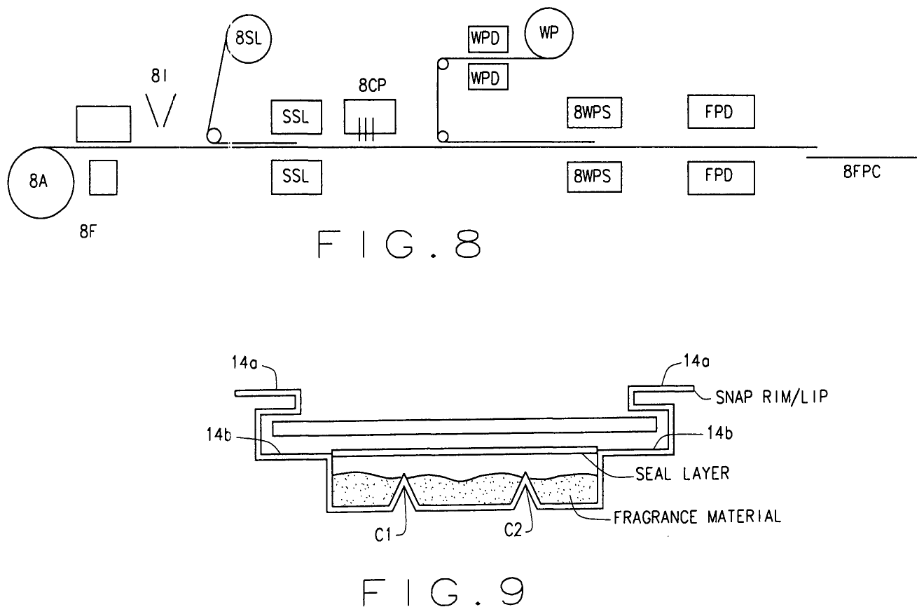

As can be seen in FIG. 8, therein is disclosed the process for making the liquid air freshener/device of this invention. In its sequented operations, a roll of the heat formable thermoplastic material, is provided at A. The thermoforming machinery is located at F, for forming the configuration of the container shell 2. At I, a supply of the fragrance material, in liquid form, is injected into the formed container shell. Then, the seal layer SL is applied to the container shell, above the fragrance material, and a heat seal is applied to secure the seal layer to the flange b, of FIG. 7, of the container shell. Then, the fragrant material paper ply CP is added into the container shell, above the seal layer, as can be noted. Then, the window ply is unrolled and applied to the top of the container, being applied to its layer a, as also explained in FIG. 7.

Then the die-cuts the windows out of the window ply, at WPD. These are the apertures 8, as previously reviewed. Then, the window ply is also heat sealed to its particular flange a. The final product is individually die-cut from the web of material, as noted at the step FBD, and this provides the finished product for collection onto the conveyor or bin collector FPC, as can be understood. This explains the process of formation, and assembly, of the entire liquid air freshener/device, of this invention.

FIG. 9 shows a modification to the invention, and in this particular instance, there may be a couple of sharp protrusions provided, at c1 and c2, to function in the manner as previously explained with respect to the embodiment of FIG. 6. Furthermore, the container may be formed with multiple rims, or lips, as at 14, and these are used for holding the window ply in place, upon the top of the fragrance material formulated container, and its carrier.

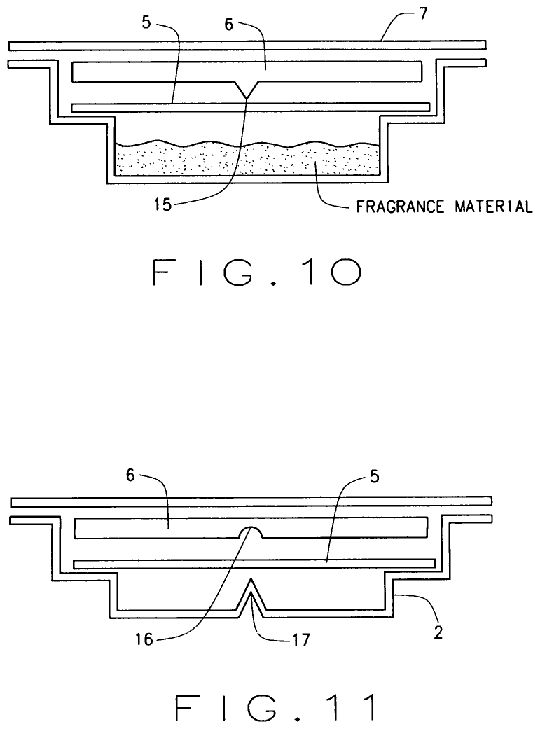

FIG. 10 provides an alternative perforation means for the invention. As noted, the protrusion 15 is integrally formed upon the fragrance carrier 6, as previously reviewed, and the window ply, and the fragrance carrier 6, can be forced downwardly, for braking the seal layer 5, as previously reviewed. Thus, as can be understood, the seal layer can be pierced from either direction, either from below, as previously reviewed, or from above, as noted in FIG. 10.

A further alternative perforation means can be seen in FIG. 11. In this embodiment, the paper ply forming the fragrance carrier, as at 6, has a recess or hole 16 provided through it, or applied to its under surface. Then, this facilitates the application of the sharp protrusion 17 when forced upwardly, by pressure applied to the bottom of the container 2, to assure that the seal ply 5 gets pierced, to initiate the operations of the air freshener.

As previously referred to, the container can be made in other configurations, such as round or oval shaped, and can be made of the injection molded polymer method, even formed of wood, metal, or other materials. And while sealing is the preferred means for attaching the various seal layers, window layers, and the like, to the container, other means are easily apparent to those skilled in the trade for use for applying these layers to the formed container. For example, as previously reviewed, the invention can utilize adhesive sealing, sonic radio frequency sealing, or even mechanical attachments, as was noted in FIG. 9.

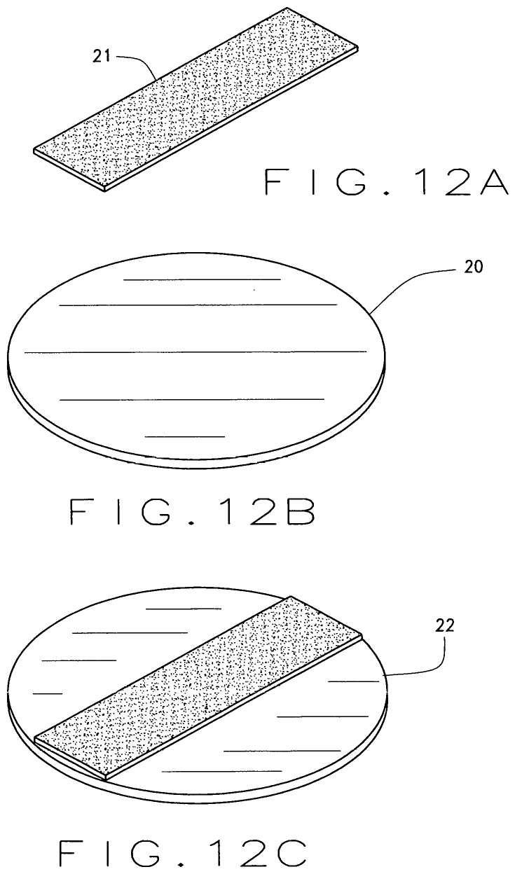

FIG. 12 A through C shows a wicking system enhancement, wherein the seal layer 20 may have a coating or strip of wicking material, as noted at 21, applied to its bottom, as noted at 22, so that when the seal layer is pierced, by the sharp protrusion, it enhances the flow of the liquid material to and through the seal layer, and into the absorbent layer, as previously identified, to provide not only an enhancement in the flow of the liquid material within the functional freshener or related device, and to provide a means for controlling the rate of flow of such material within the device, during its usage and application. As previously reviewed, it is the concept of this invention to provide a liquid air freshener, but one that is formed within an apparatus that can be also used for odor control, disseminate an insect repellent, even an insecticide, or even for therapeutic or medical applications, in the usage of this device.

Variations or modifications to the subject matter of this invention may occur to those skilled in the art upon review of the disclosure as provided herein. Such variations, if within the spirit of this invention, are intended to be encompassed within the scope of any claims to patent protection issuing herein. The definition of the invention as set forth in the specification, and its depiction in the drawings, are set forth for illustrative purposes only.

What is claimed is:

1. A liquid air freshener or device to deliver ingredients from a liquid into a vapor state, comprising:
a thermoformed container, said thermoformed container having a base, said base provided for holding a volume of liquid air freshener, a seal layer secured to the container and arranged above the disposed fragrance material, an absorbent layer provided above the seal layer, which when exposed to the fragrance material, is infused with the same for dissemination of its scent or ingredients, and a window ply provided upon the top of the container; and
a sharp protrusion operatively associated with the container and its various layers and when forced against the seal layer, piercing the same, to allow for exposure of the liquid fragrance material or ingredients to the absorbent layer to infuse it with the liquid fragrance material or ingredients and provide for its dissemination through the window ply to the surrounding atmosphere.

2. The liquid air freshener of claim 1, wherein the sharp protrusion is integrally formed in the base of the container, and when forced upwardly, through the container fragrance material, is capable of piercing the seal layer to initiate dissemination of its fragrance material or ingredients.

3. The liquid air freshener of claim 1, wherein said absorbent layer has a sharp protrusion extending downwardly, and when the absorbent layer is forced downwardly, piercing the seal layer, to allow for the migration of the fragrance material into the absorbent layer and dissemination of its scent through the window ply and into the surrounding atmosphere.

4. The liquid air freshener of claim 1, wherein said absorbent layer comprises paper.

5. The liquid air freshener of claim 4, wherein said paper absorbent layer is non-woven.

6. The liquid air freshener of claim 1, wherein the sharp protrusion means comprises more than one sharp protrusion, for separate piercing of the seal layer to allow for a gradual release of the fragrance material into the absorbent layer during prolonged usage of said freshener.

7. The liquid air freshener of claim 1, and including said thermoformed container having an integrated flange, and said window ply secured to said container flange.

8. The liquid air freshener of claim 1, and including a tab integrally formed with the container flange, and provided for suspension of the air freshener during usage.

9. The liquid air freshener of claim 1, wherein said container is formed having a series of levels that provide a base for adherence of the seal layer at one level, and the window ply at a top level of the formed container.

10. The liquid air freshener of claim 2, wherein said absorbent layer having one of a recess or hole provided therein, for accommodating the insertion of the sharp protrusion of the container base therein to assure piercing of the seal layer during initiation of usage of the air freshener.

11. The liquid air freshener of claim 1, wherein said thermal formed container having an upper flange, and said window ply connecting with said container flange.

12. The liquid air freshener of claim 11, wherein said window ply having a series of perforations provided therethrough.

13. The method of forming a liquid air freshener, including providing a roll of heat formable thermoplastic material, applying the thermoplastic material upon thermo forming machinery to process the thermoplastic material to form the configuration of a container shell, supplying a quantity of fragrance material into the formed well of the container shell, providing a seal layer upon the formed container shell to seal in the fragrance material, utilizing a heat seal to secure the seal layer to the container shell, applying a paper ply to the container shell above the seal layer, then applying a window ply having a series of perforations onto the top of the container, above the paper ply, there being at least one sharp protrusion integrally provided upon the base of the formed container shell, such that when pressure is applied to the base of the container shell the integral protrusion pierces the seal layer thereby allowing the fragrance material to saturate the paper ply and to disseminate its fragrance aroma through the window ply into the ambient atmosphere to furnish the liquid air freshener.

14. A liquid air freshener or device to deliver ingredients from a liquid into a vapor state, comprising:
a thermoformed container, said thermoformed container having a base, said base provided for holding a volume of liquid air freshener, or other liquid means, a seal layer secured to the container and arranged above the disposed fragrance material, an absorbent layer provided above the seal layer, which when exposed to the fragrance material, is infused with the same for dissemination of its scent or ingredients, and a window ply provided upon the top of the container; and
a sharp protrusion operatively associated with the container and its various layers and when manually forced against the seal layer, piercing the same, to allow for exposure of the liquid fragrance material or ingredients to the absorbent layer to infuse it with the liquid fragrance material or ingredients and provide for its dissemination through the window ply to the surrounding atmosphere.

15. The liquid air freshener of claim 14 wherein the absorbent layer may comprise paper, paper board, polymer, thin wood, and related materials.

16. The liquid air freshener of claim 15 wherein the window ply may include a series of perforations, slits, openings, shaped openings, in order to regulate the dissemination of the fragrance material or other ingredients from the container.

17. The liquid air freshener of claim 16, and including a wicking means positioned between the liquid air freshener and the absorbent layer to enhance the control of the rate of absorption of the liquid material during usage of the device.

18. A liquid delivery system for providing air freshening, odor elimination, as an insect or animal attractant or repellent, comprising:
a thermal formed container, said thermal formed container having a base, said base provided for holding a volume of said liquid, a seal layer secured to the container and arranged above the disposed liquid material; and
a sharp profusion operatively associated with a container and its various layers and when forced against the seal layer, piercing the same to allow for exposure of the liquid material to the absorbent layer to infuse it with the liquid material and provide for its dissemination out of the container and into the surrounding atmosphere.

19. The liquid delivery system of claim 18 showing wicking material operatively associated with the seal layer and to enhance the transfer of liquid material to the absorbent layer in preparation for usage of said system.

20. The liquid delivery system in 19, wherein said wicking material may be one of woven or non-woven material, cotton, or related wicking material to enhance the flow of the said liquid material to the said absorbent layer.
